(19) United States
(12) Patent Application Publication
(10) Pub. No.: US 2016/0038410 A1
(43) Pub. Date: Feb. 11, 2016

(54) ENCAPSULATION OF PHARMACEUTICALS FOR TASTE MASKING IN CHEWABLE TABLETS

(71) Applicant: BOSTON THERAPEUTICS, INC., Manchester, NH (US)

(72) Inventor: David Platt, Newton, MA (US)

(21) Appl. No.: 14/775,113

(22) PCT Filed: Mar. 14, 2014
(86) PCT No.: PCT/US14/27243

§ 371 Date: Sep. 11, 2015

Related U.S. Application Data
(60) Provisional application No. 61/789,612, filed on Mar. 15, 2013.

(51) Int. Cl. A61K 9/00; A61K 9/28

(52) U.S. Cl. CPC A61K 9/0056; A61K 9/2866

(57) ABSTRACT

The present disclosure relates to the use of polymers to coat bitter-tasting active pharmaceutical ingredients in a manner that masks the bitter taste of these compounds. Taste masked pharmaceutical formulations in which the particles of pharmaceutically active ingredients are coated with polymers or ion exchange resins are disclosed. The formulations provide taste masked pharmaceutical formulations in which the rapid disintegration of tablets is preserved. A method for preparing such coated particles in a fluidized bed coating process is disclosed. The polymer coating may include a combination of low molecular and high molecular weight water in-soluble polymers, plasticizer and fillers, which provides for a chewable dosage form having a pleasing taste thereby improving patient compliance.

### CROSS REFERENCES TO RELATED APPLICATIONS

This application claims the benefit of U.S. Provisional Patent Application Ser. No. 61/789,612, entitled: “ENCAPSULATION OF PHARMACEUTICALS FOR TASTE MASKING IN CHEWABLE TABLETS,” filed Mar. 15, 2013, the content of which is incorporated herein by reference in its entirety.

### FIELD

The present disclosure relates to pharmaceutical dosage forms providing an oral taste masking drug delivery system for active pharmaceutical ingredients.

### BACKGROUND

Bitter tasting pharmaceutically active ingredients are particularly difficult to render palatable when placed in oral tableted dosage forms. Much research and formulation techniques have been employed in the art to attempt to mask the taste of bitter or off tasting pharmaceuticals without retarding the physiological availability and/or activity of the bitter tasting active ingredients.

Well known methods for taste masking generally have involved coating of the particles of the active ingredient and/or the tablet containing such active ingredient. Unfortunately, with various coating materials or combinations of coating materials many of these approaches provide coating materials having limited water solubility and are therefore applied from organic media. On the one hand, the more water soluble such coatings are the less effective they are in taste masking, however, on the other hand the less water soluble they are the more they tend to retard the physiological availability and activity of the active ingredient.

In order to achieve both rapid disintegration and taste masking it has been necessary to use both a coating and a dis-integrant or super dis-integrant in tablet formulations. Unfortunately, this approach is extremely costly in requiring both a coating step and the addition of relatively costly dis-integrants.

### SUMMARY

The present disclosure relates to the use of manufacturing methods to coat bitter or off tasting active pharmaceutical ingredients in a manner that masks the bitter or off taste of these ingredients. According to the disclosure, taste masked pharmaceutical formulations contain particles of pharmaceutically active ingredients that are coated with polymers, ion exchange resins or granulated in a manner to reduce surface area. These approaches provide masked pharmaceutical ingredients, in which rapid disintegration of tablets is preserved.

In one illustrative embodiment, it is contemplated that a method for preparing such coated ingredient particles is accomplished by a fluidized bed coating process as known in the art.

In a further illustrative embodiment, an effective method of preparing a taste masked pharmaceutical formulation is provided that is less costly for achieving taste masking while assuring prompt physiological availability of the active ingredient.

In another illustrative embodiment, a pharmaceutical formulation is provided utilizing polymeric coatings in combination with ion exchange resins to reduce, if not substantially eliminate the undesirable taste of bitter pharmaceuticals in tableting formulations.

In yet a further illustrative embodiment, a pharmaceutical formulation is provided wherein the particle size of the active ingredient and the method used to coat the active ingredient provide characteristics that allow granulation techniques to decrease surface area of bitter pharmaceutical compounds. These findings are particularly surprising and unexpected in view of the fact these approaches have not heretofore been used as a taste masking techniques.

In a further illustrative embodiment, according to the disclosure a pharmaceutical formulation provides a chewable tablet form that includes a mannan component and possibly other taste masking and/or flavor ingredients.

In yet a further illustrative embodiment, tablets according to the disclosure have polymer coatings.

In another illustrative embodiment, tablets according to the disclosure are coated with ion-exchange resins.

In a further illustrative embodiment, the tablet is encapsulated within a lipid matrix.

In one illustrative embodiment according to the disclosure, the formulation masks the bitter taste of active pharmaceuticals ingredients, such as metformin. It is contemplated within the scope of the disclosure that due to its cationic nature, active pharmaceutical ingredients, such as metformin, can be complexed to an ion-exchange resin suppressing the bitter taste of metformin. Additionally, due to binding properties of the ion-exchange resins release profiles of the active pharmaceutical ingredients, such as metformin and the like, may achieve superior clinical pharmacokinetics. It is further contemplated within the scope of the disclosure that ion-exchange resins may be useful in masking the taste of other active pharmaceutical ingredients.

In a further illustrative embodiment according to the disclosure, active pharmaceutical ingredients are coated with polymers. The polymeric coating acts as a physical barrier to minimize interactions of the pharmaceutical ingredient with taste receptors. It is contemplated within the scope of the disclosure that insoluble polymers for taste masking may include cellulose esters, PVP-vinyl acetate, ethyl and hydroxyethyl cellulose and the like. Possible application techniques may be those known in the art, including but not limited to spray-dry coating and freeze-dry encapsulation.

In yet another illustrative embodiment, a granulation process is used to reduce an effective surface area of the bitter component, such as metformin. According to the disclosure, granulation could be performed utilizing dry, wet, and melt methods.

In another illustrative embodiment, adsorbates such as vegum, bentonite, silica gels, and silicates are often used together with other taste masking technologies to help trapping the drug and aid sustained/delayed release. It is contemplated within the scope of the disclosure that adorbates can be used alone or in combination with polymer coatings, granulation and ion-exchange resins.

### BRIEF DESCRIPTION OF THE DRAWINGS

Embodiments of compositions, systems, and methods are illustrated in the figures of the accompanying drawings which are meant to be exemplary and not limiting, in which like references are intended to refer to like or corresponding parts, and in which:

FIG. 1 illustrates a block flow diagram of an exemplary method of taste masking a pharmaceutical according to an aspect of the present disclosure;

FIG. 2 illustrates a block flow diagram of an exemplary method of taste masking a pharmaceutical according to another aspect of the present disclosure;

FIG. 3 illustrates a block flow diagram of an exemplary method of performing glycosyl linkage analysis according to an aspect of the present disclosure;

FIG. 4 illustrates exemplary results of the linkage analysis for Sample A;

FIG. 5 illustrates exemplary results of the linkage analysis for Sample B;

FIG. 6 illustrates a block flow diagram of an exemplary method of performing glycosyl composition analysis according to an aspect of the present disclosure;

FIG. 7 illustrates exemplary results of the glycosyl composition analysis for Samples A and B;

FIG. 8 illustrates exemplary results of performing size exclusion chromatography for Sample A; and

FIG. 9 illustrates exemplary results of performing size exclusion chromatography for Sample B.

### DETAILED DESCRIPTION

Detailed embodiments of the present taste masking pharmaceutical delivery system is disclosed herein, however, it is to be understood that the disclosed illustrative embodiments are merely exemplary, which may be embodied in various forms. Therefore, specific functional details disclosed herein are not to be interpreted as limiting, but merely as a basis for the claims and as a representative basis for teaching one skilled in the art to variously employ the pharmaceutical delivery system disclosed herein.

The present disclosure relates to novel oral delivery systems for bitter tasting active pharmaceutical ingredients providing in one illustrative embodiment patient compliant chewable dosage forms having either instant release or sustained release formulations that mask bitter tasting pharmaceuticals.

According to the disclosure, a chewable formulation comprising an active pharmaceutical agent in solubilized form with taste masking coatings are envisioned.

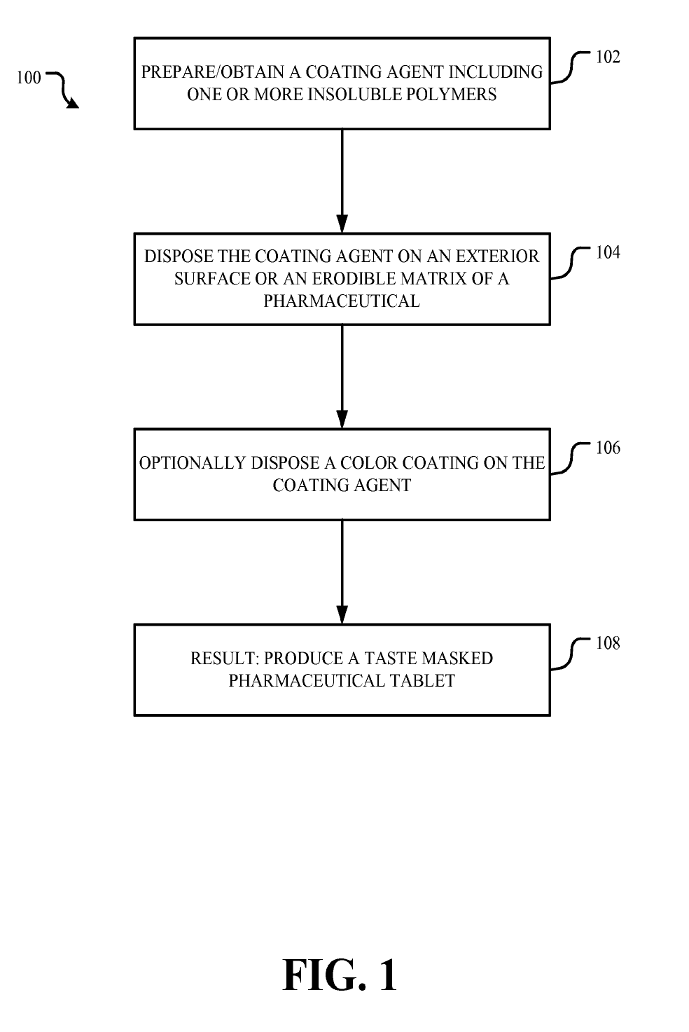

In an illustrative embodiment, bitter tasting pharmaceuticals are coated with insoluble polymers. A method 100 of taste masking a pharmaceutical according to an aspect of the present disclosure is described with reference to FIG. 1. As illustrated, a coating agent containing one or more insoluble polymers is prepared or obtained, illustrated as block 102. The coating agent is disposed on an exterior surface of the pharmaceutical or an erodible matrix of the pharmaceutical, as illustrated in block 104. The coating agent acts as a physical barrier to minimize interactions of the pharmaceutical/drug with taste receptors. The coating agent may be coated on the active pharmaceutical ingredient such that the polymer(s) are present in an amount of about 10 to about 50 percent by weight and more particularly about 0.5 to about 30 percent by weight of the pharmaceutical formulation. Optionally, a color coating may be applied to the coating agent, illustrated as block 106. The result is a taste masked pharmaceutical 108.

It is contemplated within the scope of the disclosure that insoluble polymers for taste masking may include, but are not limited to, cellulose esters, PVP-vinyl acetate, ethyl and hydroxyethyl cellulose and the like. High molecular weight and low molecular weight hydrophobic polymers are contemplated within the scope of the disclosure. Optionally, the pharmaceutical compositions disclosed herein may be further coated with a functional coating comprising combination of low molecular and high molecular weight water insoluble polymers, plasticizer and fillers, which provides for taste masking.

The drug delivery system according to the disclosure contains at least one active pharmaceutical ingredient; however, it's contemplated within the scope of the disclosure that one or more active pharmaceuticals can be in combination. It is further contemplated that the active pharmaceuticals can be within an erodible matrix providing sustained release of active pharmaceuticals. The erodible matrix comprising a mixture of low molecular weight and high molecular weight hydrophilic polymers enables controlled erosion providing sustained release of an active pharmaceutical agent. The erodible matrix can be coated with the polymeric coating according to the disclosure.

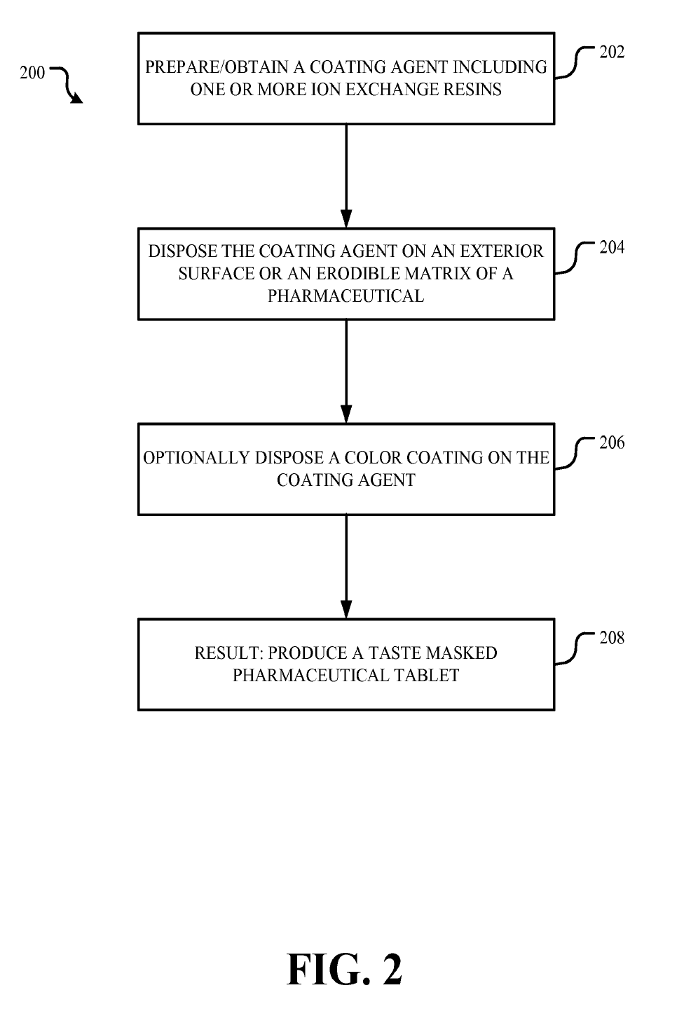

The composition of pharmaceutical formulations according to the disclosure may be optionally coated with an ion-exchange resin. In an illustrative embodiment, an ion exchange resin is used to taste mask the pharmaceutical ingredient. A method 200 of taste masking a pharmaceutical ingredient according to this embodiment is described with reference to FIG. 2. As illustrated, a coating agent containing one or more ion exchange resins is prepared or obtained, illustrated as block 202. Similar to the method described with reference to FIG. 1 above, the coating agent is disposed on an exterior surface of the pharmaceutical or an erodible matrix of the pharmaceutical 204. Optionally, a color coating may be applied to the coating agent, illustrated as block 206. The result is a taste masked pharmaceutical 208. It is contemplated within the scope of the disclosure that coloring agents may be incorporated into the coating agents or applied in a separate layer over the coating agents.

The active pharmaceutical ingredient in the pharmaceutical formulations according to the disclosure could be any active pharmaceutical ingredient that is suitable for use in instant or sustained-release formulations. Exemplary active pharmaceutical ingredients include but are not limited to: antihypertensive drugs such as isradipine, nifedipine, doxazocin, amosulralol, felodipine, lercanidipine, lecidipine, nicardipine, fosinopril, imidaprile, clizapril, perindopril, losartan, irvesartan, candesartan; steroidal drugs; anti-diabetic drugs such as metformin, gliclazide, glimepirideand glipizide, isradipine and nifedipine. The pharmaceutical ingredient may be used in the range of about 0.5-60 wt %, preferably about 1 to 30%. It is contemplated within the scope of the disclosure that the active pharmaceutical ingredient may be in combination with one or more other active pharmaceutical ingredients.

The pharmaceutical formulations according to the disclosure may also optionally further comprise water soluble low molecular weight and high molecular weight polymers. Water soluble low molecular weight and high molecular weight polymers can include but not be limited to: saccharides, cellulose derivatives, gums, vinyl polymers, acrylates, polyethylene derivatives, etc. and mixtures thereof.

The pharmaceutical formulations according to the disclosure may optionally further include hydrophilic polymer can include such as saccharides, dextrin, polydextrin, dextran, pectin, pectin derivatives, alginate, polygalacturonic acid, xylan, arabinoxylan, arabinogalactan, starch, hydroxypropyl starch, amylose, amylopectin, and the like.

The pharmaceutical formulations according to the disclosure may further optionally include cellulose derivatives such as hydroxypropylmethylcellulose, hydroxypropylcellulose, hydroxymethylcellulose, hydroxyethylcellulose, methylcellulose, sodium carboxymethylcellulose, cellulose acetate, hydroxyethylmethylcellulose and the like.

The pharmaceutical formulations according to the disclosure may also optionally include guar gum, locust bean gum, tragacantha, carrageenan, acacia gum, arabia gum, gellan gum, and the like. It is envisioned that proteins such as gelatin, casein and the like may be further optionally utilized.

The pharmaceutical formulations according to the disclosure may also additionally include polyvinyl derivatives such as polyvinyl alcohol, polyvinylpyrrolidone, polyvinylacetaldiethylaminoacetate, and the like may be optionally utilized.

The pharmaceutical formulations according to the disclosure may further optionally include polymethacrylate copolymers, such as poly(butyl methacrylate, (2-dimethylaminoethyl) methacrylate, methylmethacrylate) copolymer, polymethacrylic acid, methylmethacrylate) copolymer, poly(methacrylic acid, ethylacrylate) copolymer, and the like.

The pharmaceutical formulations according to the disclosure may additionally include polyethylene derivatives such as polyethylene glycol, polyethylene oxide, are contemplated within the scope of the disclosure.

The pharmaceutical formulations according to the disclosure may also include carboxyvinyl polymers such as carbomer, and the like are envisioned. Preferable cellulose ether derivatives such as hydroxypropylmethylcellulose, is contemplated within the scope of the invention.

According to the disclosure the low molecular weight polymer may be used in the range of about 5 to about 70 wt % and preferably about 10 to about 40 wt %. The high molecular weight polymer may be used in the range of about 5 to about 70 wt % and preferably in the range of 10 to 40 wt %.

The hydrophobic polymers used according to the disclosure include, but are not limited to, cellulose acetate phthalate, hydroxypropyl methyl cellulose phthalate, hydroxypropyl methyl cellulose acetate succinate, methacrylate copolymers, shellac, zein, poly vinyl acetate phthalate, more preferably hydroxyl propyl methyl cellulose phthalate and hydroxylpropylmethyl cellulose acetate succinate, most preferably hydroxylpropylmethyl cellulose acetate succinate is used. The pH sensitive enteric polymer can be used in a range from about 0.5 to 30 wt %, preferably about 1 to 10 wt %.

The pharmaceutical formulations according to the disclosure may additionally include water soluble fillers that include, but are not limited to, carbohydrates such as mannitol, sorbitol, arabinose, ribose, xylose, glucose, fructose, mannose, galactose, sucrose, maltose, lactose, raffinose, high molecular weight polyethylene glycols, electrolytes such as sodium chloride, sodium dihydrogen phosphate, sodium and potassium bicarbonates etc. More preferably carbohydrates and its derivatives. Most preferably lactose or manitol can be used. Water soluble fillers can also be used in range from about 5 to about 75 wt % and preferably about 20 to about 60 wt %.

The pharmaceutical formulations according to the disclosure may also optionally include water insoluble fillers such as cellulose and its derivatives, calcium carbonate, magnesium carbonates, magnesium oxides, dicalcium phosphate, starch and its derivatives can be used. More preferably cellulose and its derivatives are used and most preferably micro crystalline cellulose is used. It is contemplated within the scope of the disclosure that water insoluble fillers can be used in range from about 5 to about 75 wt % and preferably about 10 to about 40 wt %.

The pharmaceutical delivery system for bitter tasting drugs disclosed herein may comprise a coating of a water soluble polymer, which enables taste masking of active agent. Examples of water soluble polymers could be but are not limited to low viscosity grade methylcellulose, hydroxypropylmethylcellulose, hydroxyethylcellulose, hydroxypropylcellulose, hydroxylethylmethylcellulose, carboxymethylcellulose, sodium carboxymethylcellulose, polyvinyl pyrrolidone and combinations thereof. More preferably cellulose ether derivatives and most preferably low molecular weight hydroxyl propyl methyl cellulose can be used. It is contemplated within the scope of the disclosure that water soluble polymers can be used in range from about 20 to about 100 wt %, preferably about 60 to about 100 wt %.

Examples of water insoluble polymer in coating include, but are not limited to, ethyl cellulose and its derivatives, cellulose acetates and vinyl polymers. More preferably cellulose derivatives and most preferably low molecular weight ethyl cellulose and its derivatives can be used. It is contemplated within the scope of the disclosure that water insoluble polymers can be used in a range from about 1 to about 30 wt %, preferably about 1 to about 15 wt %.

The pharmaceutical delivery system according to the disclosure may also include other additives like titanium dioxide, talc, fillers and plasticizer like dibutyl sebacate, triethylcitrate, polyethylene glycol derivatives, castor oil and the like. It is contemplated within the scope of the disclosure that the coating could be applied from about 2 to about 15% weight gain, preferably about 3 to about 10% weight gain.

The pharmaceutical delivery system for bitter tasting drugs according to the disclosure may additionally contain color coating(s) to provide a more elegant pharmaceutical formulation. It is contemplated within the scope of the disclosure that the color coating can be applied in the range of about 1 to about 10% weight gain preferably about 2 to about 5% weight gains.

In addition to above excipients, tablet formulations according to the disclosure may optionally contain solubilizers like sodium lauryl sulfate, vitamin E derivatives, poloxamers, tween 80, low molecular weight cellulose derivatives, low molecular weight pyrollidone derivatives can be used, and preferably sodium lauryl sulphate and poloxamer and most preferably sodium lauryl sulphate can be used.

In addition, lubricants like talc, magnesium stearate, calcium stearate, zinc stearate, lauryl sulfate, hydrogenated vegetable oil, sodium benzoate, sodium stearyl fumarate, glyceryl monostearate and glidants, antiadherent and other standard tableting excipients known in art can be used in the tablet formulation. The formulation would be designed as a compressed tablet or caplet by standard tableting techniques, and coated using standard coating equipment and methods known in the art such as coating pans, automatic coater or fluid bed coater.

### EXAMPLES

Aspects of the disclosure are further described in detail as in the following examples. However, the following examples are not intended to limit the scope of the disclosure to the precise details of methodology or construction set forth below. Practical and illustrative embodiments are illustrated and described in the following examples. However, it should be appreciated that those skilled in the art may make modifications and improvements within the spirit and scope of the present disclosure.

### Example I

### Linkage Analysis

In an illustrative example, glycosyl linkage analysis was performed on Samples A and B In general, the samples were permethylated, depolymerized, reduced, and acetylated; and the resulting partially methylated alditol acetates (PMAAs) analyzed by gas chromatography-mass spectrometry (GC-MS), for example, as described by York et at (1985) Methods Enzymol, 118:3-40.

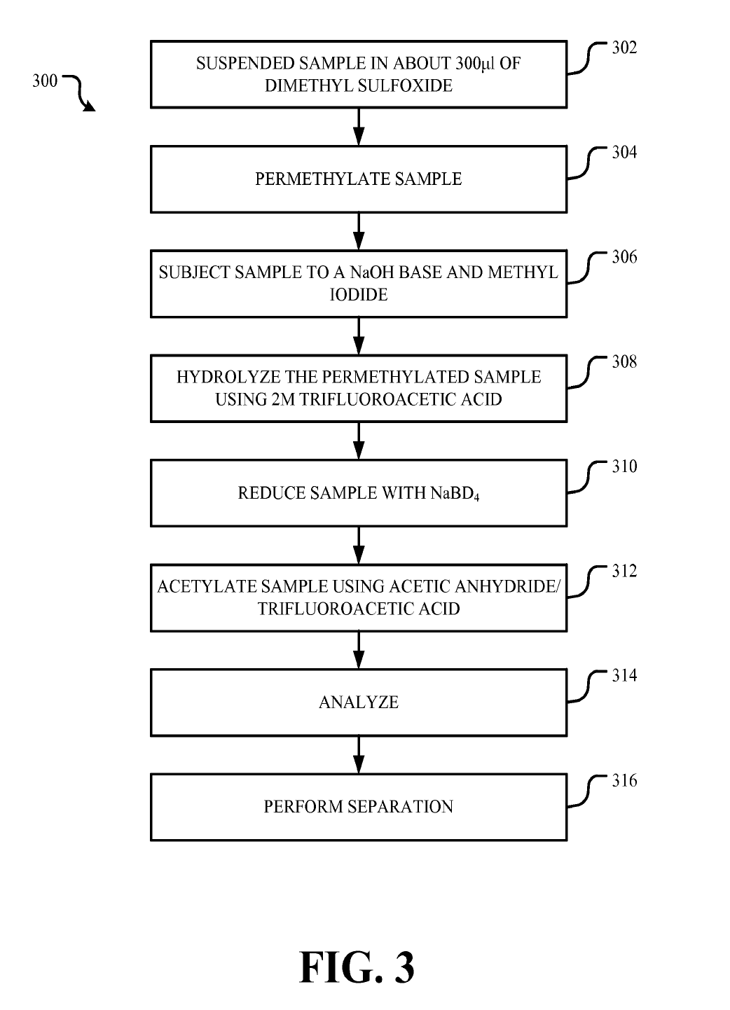

FIG. 3 illustrates a block flow diagram of an exemplary method 300 of glycosyl linkage analysis according to the present disclosure. Initially, dry samples of each of Samples A and B were suspended in about 300 μl of dimethyl sulfoxide, illustrated as block 302. The samples were then permethylated 304, for example, by the method of Ciukanu and Kerek (1984) Carbohydr. Res. 131:209-217 (treatment with sodium hydroxide and methyl iodide in dry DMSO). The samples were subjected to a NaOH base and methyl iodide was added 306. Following sample workup, the permethylated material was hydrolyzed using 2M trifluoroacetic acid 308 and then reduced with NaBD₄, 310, and acetylated using acetic anhydride/trifluoroacetic acid 312. The resulting PMAAs were analyzed on Agilent Technologies 7890 GC interfaced to a 5975C MSD mass selective detector, electron impact ionization mode) 314; and separation was performed on a 30m Supelco 2380 bonded phase fused silica capillary column 316.

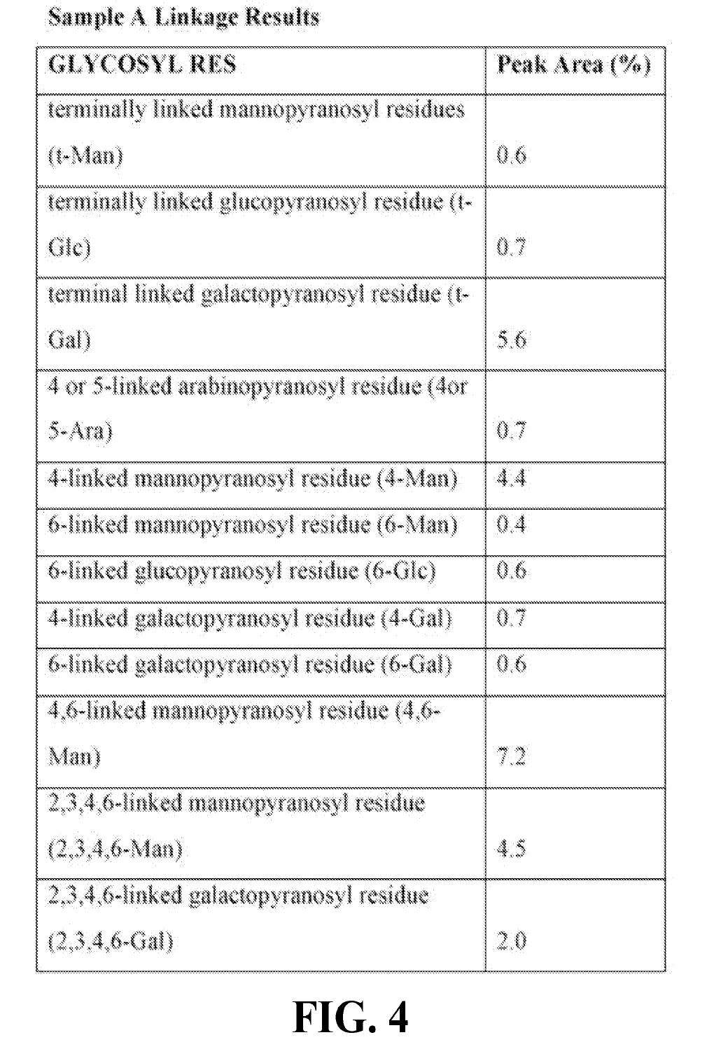

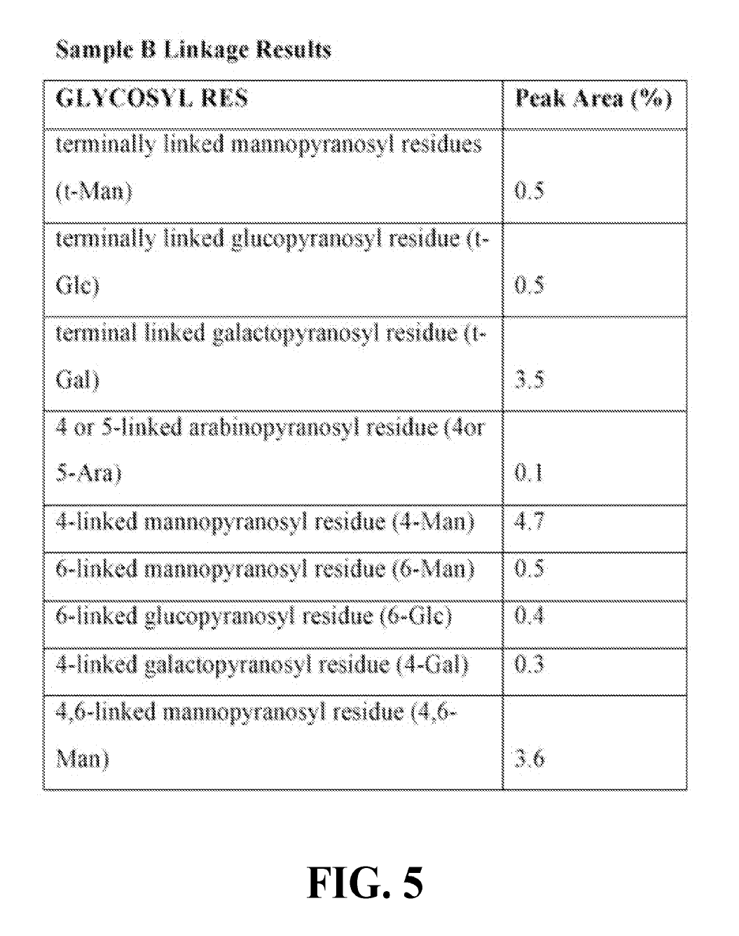

The linkage results for Samples A and B are illustrated in FIGS. 4 and 5, respectively. Referring to FIGS. 4 and 5, the linkage results indicate that both Samples A and B mainly consist of terminally linked galactopyranosyl residue (t-Gal), 4-linked mannopyranosyl residue (4-Man) and 4,6-linked mannopyranosyl residue (4,6-Man). Small amounts of other linkage residues of mannose, galactose, arabinose and glucose were also found in the Samples. Sample A contains 2,3,4,6-linked mannopyranosyl residue and 2,3,4,6-galactopyranosyl residue (2,3,4,6-Man) which indicates more branching.

### Example II

### Glycosyl Composition

In a further example, glycosyl composition analysis was performed by combined gas chromatography/mass spectrometry (GC/MS) of the per-O-trimethylsilyl (TMS) derivatives of the monosaccharide methyl glycosides produced from the Samples by acidic methanolysis.

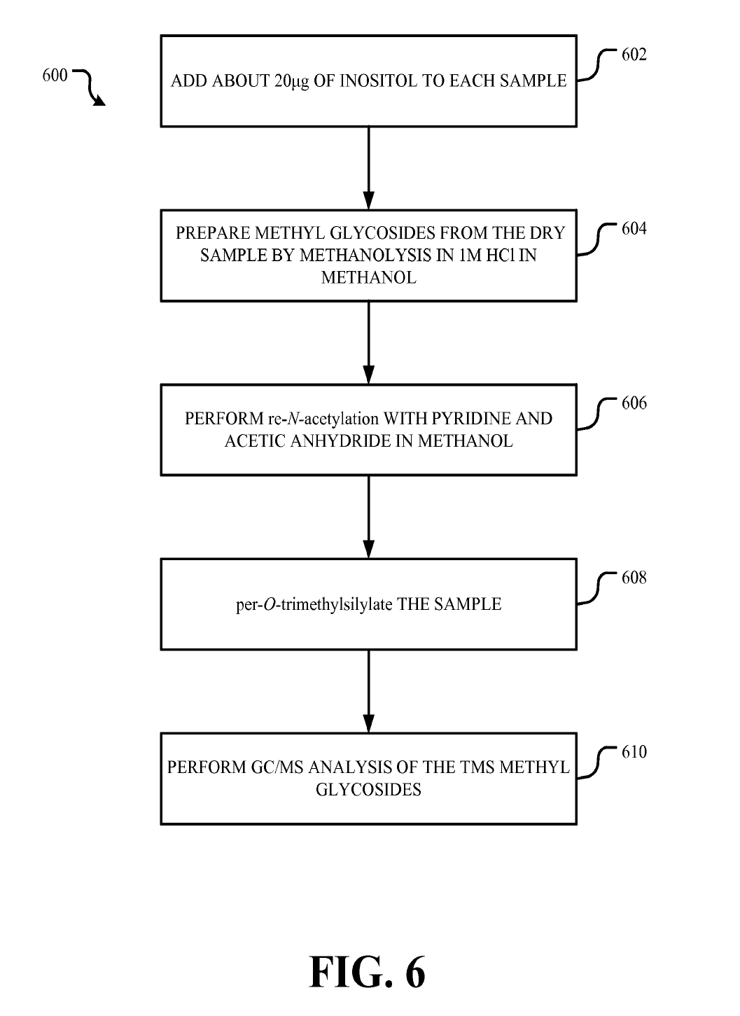

FIG. 6 illustrates a block flow diagram of an exemplary method 600 of glycosyl composition analysis according to the present disclosure. An aliquot of Sample A and Sample B was used for the analysis. About 20 μg of inositol was added to each Sample 602. Methyl glycosides were then prepared from the dry sample by methanolysis in 1 M HCl in methanol 604, followed by re-N-acetylation with pyridine and acetic anhydride in methanol (for detection of amino sugars) 606. The Samples were then per-O-trimethylsilylated 608, for example, by treatment with Tri-Sil (Pierce). These procedures were carried out as previously described, for example, in Merkle and Poppe (1994) Methods Enzymol, 230: 1-15; York, et al. (1985) Methods Enzymol, 118:3-40. GC/MS analysis of the TMS methyl glycosides was performed 610, for example, on an Agilent 6890N GC interfaced to a 5975B MSD, using a Supelco EC-1 fused silica capillary column (30 m×0.25 mm ID).

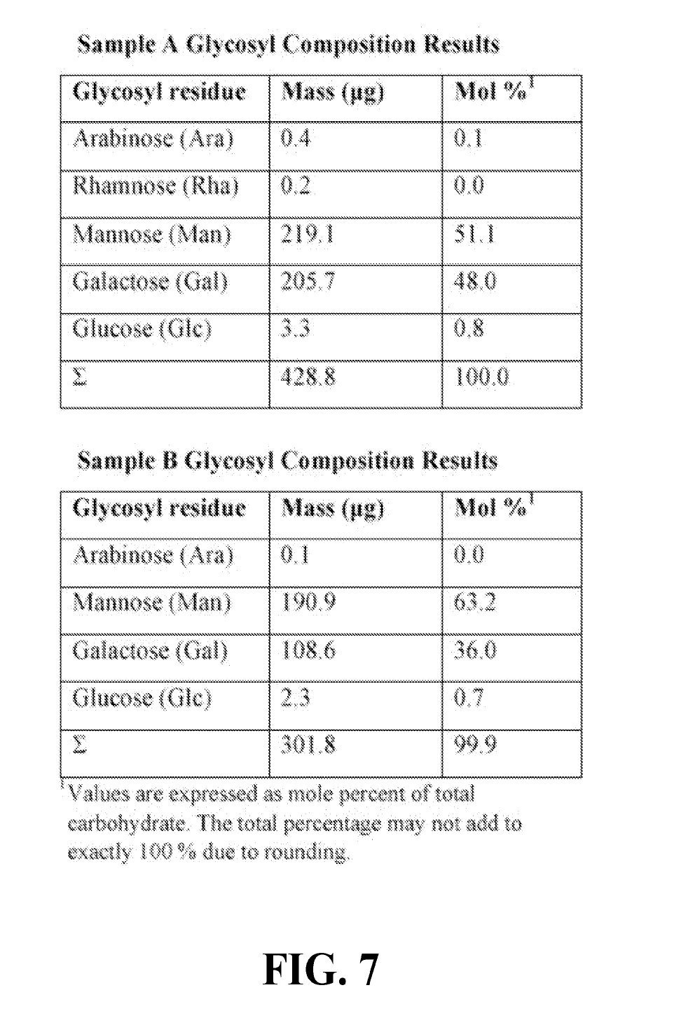

The glycosyl composition analysis results for Samples A and B are illustrated in FIG. 7. Referring to FIG. 7, the glycosyl composition analysis results demonstrate that the Samples contain mannose and galactose as the major monosaccharide residue. Other residues such as glucose and arabinose were also found in minute amount in both Samples. Sample A was also found to have a minute amount of rhamnose residue.

### Example III

### Size Exclusion Chromatography

In yet a further example, size exclusion chromatography was performed. In this example, a dilute solution of the Samples (5 mg/ml) were prepared and passed through a 0.22 μm spin filter, followed by a 100 μl injection into an HPLC. For example, the size exclusion chromatography may be performed on an Agilent 1100 HPLC system. Column: Superose 12 (GE Healthcare Life Sciences); Eluent: 50 mM Ammonium Acetate; Flow rate: 1.0 mL/min; and Detection: ELSD.

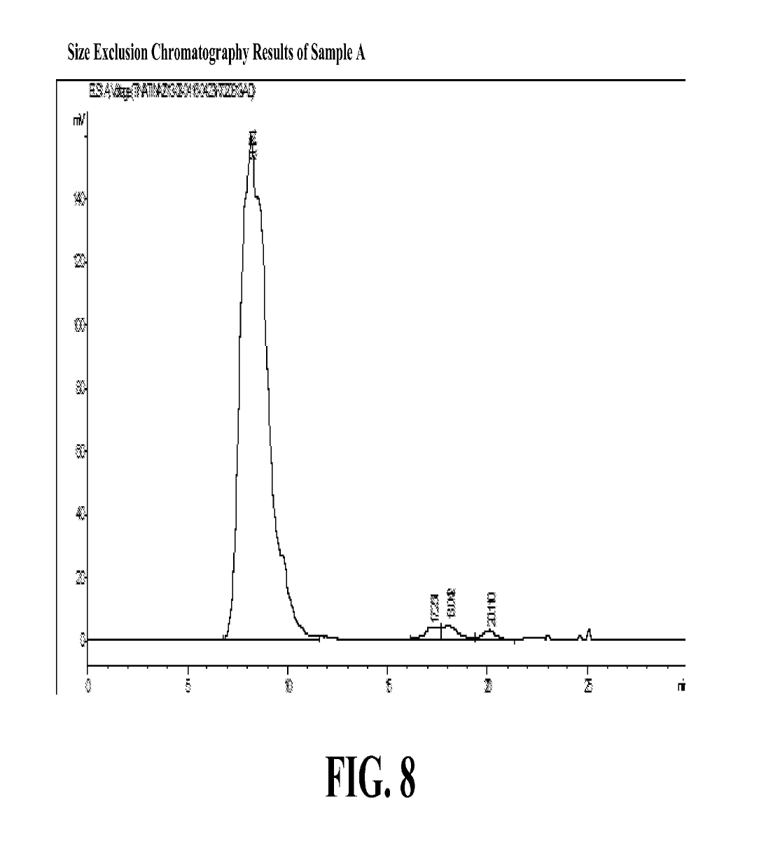

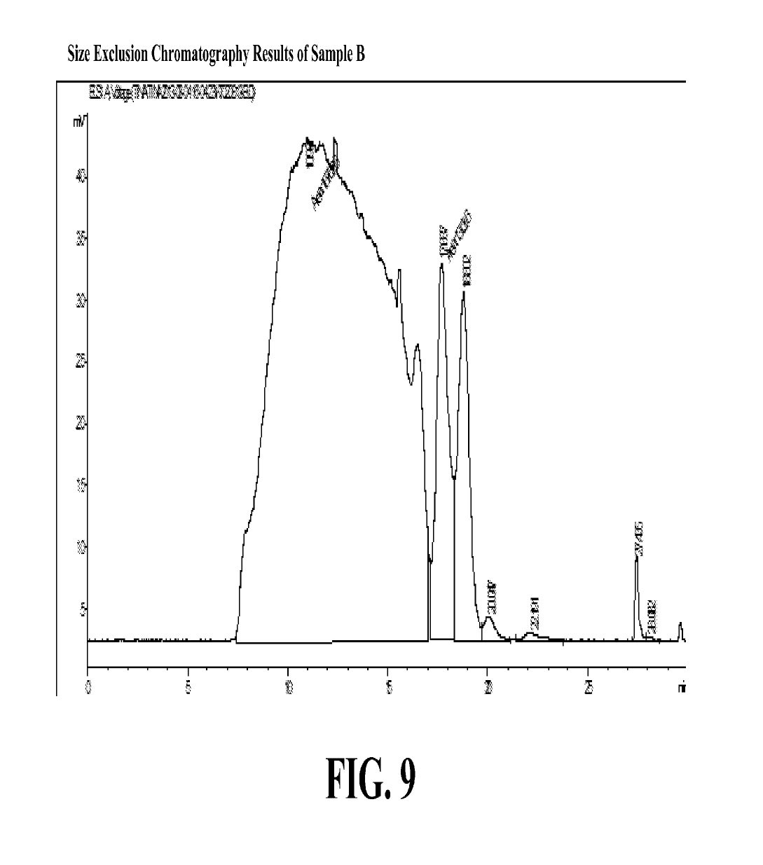

The size exclusion chromatography results for Samples A and B are illustrated in FIGS. 8 and 9, respectively, and Table 1 below.

TABLE 1
Size exclusion chromatography results for Samples A and B.

| Sample | Peak | Retention time | Estimated MW (Daltons) |
|---|---|---|---|
| A | 1 | 8.174 | 200000 |
| | 2 | 20.212 | <1000 |
| B | 1 | 8.12 | 210000 |
| | 2 | 17.1 | <1000 |
| | 3, 4, 5 | 18.8, 20.047, 27.435 | <1000 |

Referring to FIG. 8 and Table 1, the size exclusion chromatography results for Sample A indicate peak 1 at 8.174 min, which is the largest peak in Sample A, can be predicted to be around 200 KD. The other peaks in Sample A, which come later in the run, can be estimated to be very small in molecular weight. Thus, Sample A is fairly pure with only one major peak.

Referring to FIG. 9 and Table 1, the size exclusion chromatography results for Sample B indicate a broad peak starting from 7.5 to 17.1 min, which can have molecular weight predicted from about 210 KD to about <1000 KD. All other peaks which come later in the run can be estimated to be very small in molecular weight. Sample B was not completely soluble in the buffer so it was vortexed and sonicated for about over an hour to make it more soluble. The sample appears to be a complex mixture of polysaccharides with a very broad molecular weight range.

Although the compositions, systems, and methods have been described and illustrated in connection with certain embodiments, many variations and modifications should be evident to those skilled in the art and may be made without departing from the spirit and scope of the disclosure. The disclosure is thus not to be limited to the precise details of methodology or construction set forth above as such variations and modification are intended to be included within the scope of the disclosure.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A taste masked pharmaceutical composition, comprising: particles of a pharmaceutically active agent coated with polymers in an amount in the range of about 10 to 50 percent by weight of the substrate.

2. The pharmaceutical composition as set forth in claim 1, wherein the pharmaceutically active agent constitutes from about 0.5 to about 60 wt %.

3. The pharmaceutical composition as set forth in claim 1, wherein the pharmaceutically active agent is selected from the group consisting of: isradipine, nifedipine, doxazocin, amosulralol, felodipine, lercanidipine, lecidipine, nicardipine, fosinopril, imidaprile, clizapril, perindopril, losartan, irvesartan, candesartan, steroids, metformin, gliclazide, glimepirideand glipizide, isradipine and nifedipine.

4. The pharmaceutical composition as set forth in claim 1, wherein the polymer coating is a combination of hydrophobic low molecular weight polymer, and high molecular weight polymer.

5. The pharmaceutical composition as set forth in claim 4, wherein the combined polymers constitute in a range from about 0.5 to about 30 wt %.

6. The pharmaceutical composition as set forth in claim 4, further comprising a water insoluble release modulators, said release modulator is selected from the group consisting of cellulose and its derivatives, calcium carbonate, magnesium carbonates, magnesium oxides, dicalcium phosphate, starch and its derivatives and combinations thereof.

7. The pharmaceutical composition as set forth in claim 4, further comprising tableting excipients selected from the group consisting of 1-HPC, polyvinyl pyrolidone, low viscosity grade cellulose derivatives, starch and its derivatives, gelatin, gums and mixtures thereof.

8. The pharmaceutical composition as set forth in claim 4, further comprising a binder from about 0.1 to about 10 wt %.

9. The pharmaceutical composition as set forth in claim 4, further comprising standard tableting excipients selected from the group consisting of sodium lauryl sulfate, vitamin E derivatives, poloxamers, tween 80, low molecular weight cellulose derivatives and low molecular weight pyrollidone derivatives.

10. The pharmaceutical composition as set forth in claim 4, further comprising lubricants selected from the group consisting of talc, magnesium stearate, calcium stearate, zinc stearate, lauryl monostearate and glidants.

11. The pharmaceutical composition as set forth in claim 4, wherein low molecular weight hydrophobic polymer is selected from the group consisting of ethyl cellulose and its derivatives, cellulose acetates and vinyl acetate polymers.

12. The pharmaceutical composition as set forth in claim 4, wherein hydrophobic low molecular polymers constitute about 1 to about 30 wt % of the coating.

13. The pharmaceutical composition as set forth in claim 4, further comprising a plasticizer, selected from the group consisting of dibutyl sebacate, triethylcitrate, polyethylene glycol derivatives, castor oil and triethyl citrate

14. The pharmaceutical composition of claim 13, wherein said plasticizer is about 1 to 20 wt % of the coating.

15. The pharmaceutical composition as set forth in claim 4, wherein the coating layer has weight of about 2-15 wt % of the tablet weight.

16. The pharmaceutical composition as set forth in claim 14, wherein the polymer coating may further contain a color layer.

17. The drug delivery system as set forth in claim 16, wherein the color coat may contain pharmaceutically acceptable colors selected from the group consisting of ferric oxides and aluminum lakes.

18. The pharmaceutical composition as set forth in claim 14, further comprising fillers selected from the group consisting of titanium dioxide and talc.

19. The pharmaceutical composition as set forth in claim 14, further comprising plasticizers selected from the group consisting of polyethylent gycol derivatives and triethyl citrate.
